(19) United States
(12) Patent Application Publication
(10) Pub. No.: US 2020/0222128 A1
(43) Pub. Date: Jul. 16, 2020

(54) ROBOT AND METHOD FOR TREATING SURFACES

(71) Applicant: KUKA Deutschland GmbH, Augsburg (DE)

(72) Inventor: Lars-Sören Ott, Düren (DE)

(21) Appl. No.: 16/631,936

(22) PCT Filed: Jul. 13, 2018
(86) PCT No.: PCT/EP2018/069069

§ 371 Date: Jan. 17, 2020

(30) Foreign Application Priority Data
Jul. 17, 2017 (DE) 10 2017 116 004.3

(51) Int. Cl. A61B 34/30; A61B 18/20

(52) U.S. Cl. CPC A61B 34/30; A61B 2017/00769; A61B 18/201

(57) ABSTRACT

A robot includes an arm having a plurality of links, drives for moving the links, a control device for actuating the drives, and a process head connected to the arm. The control device is configured to operate the drives in a force- and/or torque-controlled manner such that the process head or a first element extending from the arm touches a surface having a structure and/or having at least one characterizing feature, and is moved along the surface with simultaneous elongation or simultaneous tightening of the surface. The process head has a radiation-emitting radiation source, and the wavelength and/or intensity of the radiation can be adjusted based on the structure and/or the at least one characterizing feature.

### CROSS-REFERENCE TO RELATED APPLICATIONS

This application is a national phase application under 35 U.S.C. § 371 of International Patent Application No. PCT/EP2018/069069, filed Jul. 13, 2018 (pending), which claims the benefit of priority to German Patent Application No. DE 10 2017 116 004.3, filed Jul. 17, 2017, the disclosures of which are incorporated by reference herein in their entirety.

### TECHNICAL FIELD

The invention relates to a robot, comprising an arm that has a plurality of links, drives for moving the links, a control device for actuating the drives and a processing head that is connected to the arm. The invention also relates to a method for machining or treating a surface that has a structure and/or at least one mark.

### BACKGROUND

Robots, i.e. multipurpose handling devices having freely programmable motion sequences, are used extensively in industry and medicine. In this case, humans and robots can perform highly sensitive tasks in close collaboration, as are done by the robot “LBR iiwa” by KUKA AG, Augsburg. By means of link torque sensors, contacts are immediately recognized and therefore dangerous collisions are ruled out.

Within the field of medicine, operations are already carried out by robots. Medical robots are also used for treating tumors. Therefore, EP 2 412 406 B1 discloses a medical robot, in which the force of the drives is adjusted in order to be able to press a sound source on a living organism with a preset force with high-intensity focused ultrasound.

Robots are likewise used to remove tattoos, such as can be found in U.S. Pat. No. 8,036,448 B2. However, skin imperfections can lead to problems when removing the tattoo.

### SUMMARY

The object of the present invention is to develop a robot and a method of the above-described type such that a surface can be machined or treated with a high degree of precision, in order to modify or remove any structures or marks.

The object is achieved by means of a robot of the above-mentioned type by the control device being designed to operate the drives so as to adjust the force and/or torque thereof such that the processing head or a first element extending from the arm touches a surface that has a structure and/or at least one mark and is moved along the surface while simultaneously stretching or simultaneously tightening the surface, that the processing head comprises a radiation source that emits radiation, and that the wavelength and/or intensity of the radiation can be influenced on the basis of the structure and/or the at least one mark.

Properties of, for example, an LBR iiwa robot by KUKA AG, are used to move the links of the robot arm by means of drives, whose force and/or torque is adjusted, such that, when it makes contact with the surface and when it moves along and on this surface, said surface is stretched or tightened, and is therefore smoothed out in the region where the radiation strikes. The regions to be struck by the radiation are therefore quasi-standardized in order to achieve reproducible results. At the same time, the properties of the structure or the mark are taken into consideration in order to treat or machine the structure or mark on the basis of the object to be achieved. In this case, a possible field of application is the removal of tattoos.

Furthermore, the invention also provides that the robot comprises an optical sensor that records the surface or a region thereof in order to be able to analyze the region to be treated or machined by means of image processing, and therefore to adjust and/or control the wavelength and/or intensity of the radiation.

It is also possible to targetedly influence the temperature of the surface or the region by the robot being provided with an apparatus that cools the surface. This can be a dispenser that delivers a coolant.

The skin is smoothed out in particular as a result of direct contact between the processing head and the surface. However, it is also possible to establish contact between the surface and an element that is assigned to the processing head, in particular such that it does not move. A relative movement, in particular a controlled relative movement, between the processing head and the element is likewise possible.

However, smoothing can alternatively or additionally also be achieved by thermal action.

Due to the inventive teaching, it is possible to automatically or possibly partially automatically machine or treat a surface or a region thereof, with optimum results being achieved when the structure or mark of the surface or region thereof is taken into account.

In one embodiment, the surface or the region on which or in which the radiation impinges is cooled or adjusted to temperatures that allow for optimum machining. In this case, the temperature influence, in particular cooling, can happen in front of the region where the radiation strikes.

In the event of cooling, it is possible for an apparatus, such as a dispenser, which delivers a coolant, to be arranged in front of the processing head or the element that extends from the arm and does not move with respect to the processing head in the direction of movement.

The fact that the movement of the arm of the robot is adjusted with regard to the force or torque ensures that further movement is prevented when too strong forces act on the robot arm, and therefore dangerous collisions with living organisms are ruled out.

The pressure that acts on the surface in order to move the robot on the surface and to stretch or tighten said surface should be between 1 KPa and 6 KPa.

Furthermore, the surface should be stretched or tightened in the region where the radiation strikes by from 0.5 percent to 2.0 percent. As a result, the required smoothing effect can be achieved in order to reproducibly treat or machine said surface.

By means of the inventive teaching, the robot arm can automatically be placed on the surface to be treated or machined and moved therealong. With the aid of optical sensors and an image processing unit in particular, signals can be generated that are used to adjust the movement of the robot arm.

During automatic operation, the orientation and position of the surface can be determined by means of the optical sensor system—alternatively or additionally by means of a tactile sensor system—and used for automatic motion planning. By means of distance sensors or tactile sensors, the movement of the surface with respect to the robot can also be recorded.

Offline path or process planning is also possible, and therefore process parameters can be varied in a spatially resolved manner.

Semi-automatic operation may also be possible. In this method, the path of the processing head is preset. The robot then travels on the preset path, in particular with constant machining parameters. Of course, it is also possible to use a sensor system that is provided in the robot, i.e. force and torque sensors in particular, to sample along the regions to be treated or a treatment path.

By means of the optical sensor system, it is possible to record the shape of the marks or structures and colors present in order to then vary the radiation to be applied on the basis of the tasks, such as removing marks.

It is also possible to apply marks to the surface that are used for automatic motion sequences. Corresponding marks allow for automatic setting off and accurate positioning of machining or treatment regions. It is conceivable to imprint special marks and use camera-based texture recognition algorithms (optical mouse principle).

Laser radiation in particular is used as the radiation, wherein direct laser sources or machining optics can be used in combination with fiber-guided laser systems. Light places can also be applied such that IPL technology can be used, for example.

When using laser sources, tunable lasers can be used. It is also possible to switch between different laser sources in order to vary wavelengths or to select the desired wavelength from a plurality of wavelengths by means of integrated switching mechanisms.

In particular, the radiation has a wavelength in the range of from UVB (300 nm-315 nm) across UVA (315 nm-400 nm) and the entire visible and NIR range, up to a possible treatment with CO₂ laser radiation at a wavelength of 10,640 nm.

In particular, a laser selected from the group of a Ruby Laser (694.3 nm), Nd:YAG Laser (1064 nm, frequency-doubled 532 nm), Er:YAG Laser (2940 nm), Alexandrite Laser (755 nm) and a CO₂ Laser (10,640 nm, 9400 nm) is used.

The disclosure also contemplates a method for machining or treating a surface having a structure and/or at least one mark by means of radiation emitted by a processing head, the processing head extending from an arm of a robot that has a plurality of links that are moved by means of drives, whose force and/or torque is adjusted, the processing head or an element assigned thereto being supported on the surface and being moved along the surface while simultaneously stretching or tightening it and the intensity and/or wavelength of the radiation emitted being adjusted and/or controlled on the basis of the structure that is recorded by means of at least one sensor and/or the at least one mark.

In particular, the motion path and speed or acceleration of the arm, from which the processing head extends, is adjusted or controlled on the basis of the structure and/or the at least one mark.

Furthermore, the wavelength and/or intensity of the radiation can be set on the basis of the pigmentation of the structure and/or the at least one mark and/or the surface, or one of several processing heads can be selected.

### BRIEF DESCRIPTION OF THE DRAWINGS

The accompanying drawings, which are incorporated in and constitute a part of this specification, illustrate exemplary embodiments of the invention and, together with a general description of the invention given above, and the detailed description given below, serve to explain the principles of the invention.

FIG. 1 is a schematic illustration of an exemplary robot in accordance with the principles of the present disclosure.

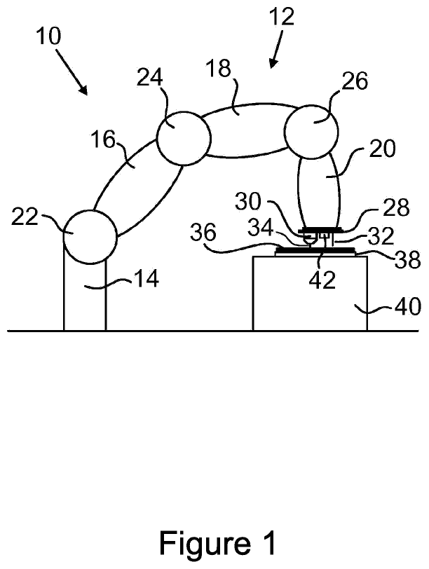

### DETAILED DESCRIPTION

In the single FIGURE, a robot 10 is shown purely schematically, which can be designed as a lightweight robot. A specific embodiment can be an LBR iiwa robot by KUKA AG. The robot 10 comprises a robot arm 12, which comprises four links 14, 16, 18, 20 in the embodiment. The links 14, 16, 18, 20 are interconnected by means of links 22, 24, 26, which are moved by drives (not shown). The drives are electric drives, which are moved by means of sensors that are integrated in the robot 10 and by a control device such that the force and torque thereof are adjusted. The arm 12 can thereby be moved with respect to six or more degrees of freedom.

The front end of the outermost link 20 comprises a flange 28, to which a processing head 30 is fastened and in which a laser beam source is integrated in the embodiment.

Furthermore, a spacer 32 extends from the flange 28. By means of the laser radiation 34 that emanates from the processing head 30, marks on a surface 36 are intended to be machined or treated, which surface is the surface of a body 38, which is positioned on a machining table 40, for example, in particular such that it does not move.

The body 38 can be an object covered in leather or hide or can be leather, hide or skin per se, for example, without the inventive teaching being restricted thereto.

Furthermore, sensors are arranged in the arm 12, which arm in particular extends from the flange 18, all of which sensors are denoted by reference sign 42. These are in particular optical sensors, by means of which the surface 36 and therefore the structure or marks of the surface 36 are recorded. The optical signals are then processed by means of an image-processing unit in order to be able to adjust or control the movement of the robot arm 12 and therefore of the laser beam 34 on the basis of the shape of the structure or mark.

In this case, it is also possible to record the color of the mark and the surface surrounding it in order to then adjust the laser radiation 34 with respect to the wavelength and/or intensity such that, once the mark has been removed, the entire surface 36 remains uniformly pigmented as desired.

In order to achieve a specific spacing from the surface 36, in the embodiment, the spacer 32 extends from the flange 28, which, when the laser radiation 34 is applied, remains in contact with the surface 36 and acts on it such that, when the arm 12 is moved, the surface 36 stretches. Therefore, smoothing can be achieved irrespective of whether the surface may be rough, which means that reproducible machining or treatment results can be achieved.

Of course, the processing head 30 itself can slide along the surface 36. The laser radiation 34 has to be adjusted accordingly.

Marks can also be made on the surface 36, which are recorded by the sensors 42 in order to set off on a path on the surface 36 that is preset by the marks.

The arm 12 is moved by means of the force and torque sensor system that is integrated in the robot 10, and that ensures that the necessary but admissible force acts on the surface 36 in order to achieve the desired stretching effect, and at the same time that damage cannot occur. This is of particular importance when a tattoo is to be removed, and therefore it ensures that dangerous amounts of force are not exerted on a body.

Semi-automatic or automatic operation is possible. For semi-automatic operation, the arm 12 travels on preset paths, in particular with constant operating parameters.

Of course, movement is also possible by sampling along the surface. For this purpose, a tactile sensor can additionally extend from the arm 12 or the link 20.

During automatic operation, the surface 36 or the region to be treated is recorded by the sensor system arranged in the robot arm 12 or by the sensors 40, in order to then move the arm 12 on the basis of existing marks or structures such that the laser beam 34 makes the desired changes to the structure or the marks to the necessary extent.

In particular, a Ruby Laser (694.3 nm), Nd:YAG Laser, Er:YAG Laser, Alexandrite Laser or a CO₂ Laser can be used as the laser. The energy density should be between 300 mJ/cm² and 10 J/cm², in particular between 300 mJ/cm² and 2 J/cm².

As the processing head slides along the surface, the pressure that acts on the surface 36 should be between 1 KPa and 6 KPa.

In the region when the radiation strikes, the surface 36 should be stretched or tightened by from 0.5 percent to 2.0 percent.

The embodiment has been described on the basis of the use of laser radiation. IPL technology can also be integrated in the processing head 30 such that treatment is carried out using high-energy flashes of light.

Instead of a radiation source provided in the processing head 30, a processing head can also be used that comprises mechanical treatment means, such as epilation units, in order to machine the surface 36.

While the present invention has been illustrated by a description of various embodiments, and while these embodiments have been described in considerable detail, it is not intended to restrict or in any way limit the scope of the appended claims to such detail. The various features shown and described herein may be used alone or in any combination. Additional advantages and modifications will readily appear to those skilled in the art. The invention in its broader aspects is therefore not limited to the specific details, representative apparatus and method, and illustrative example shown and described. Accordingly, departures may be made from such details without departing from the spirit and scope of the general inventive concept.

What is claimed is:

1-12. (canceled)

13. A robot for treating a surface that has at least one of a structure or at least one mark, the robot comprising:
an arm having a plurality of links and a plurality of drives for moving the links;
a processing head connected to the arm and comprising a radiation source that emits radiation; and
a control device configured to actuate the drives;
the control device configured to operate the drives so as to adjust forces and/or torques applied by the drives such that the processing head or a first element that extends from the arm touches the surface and is moved along the surface while simultaneously stretching or tightening the surface;
wherein at least one of a wavelength or intensity of the emitted radiation is adjusted based on at least one of the structure or the at least one mark.

14. The robot of claim 13, wherein the arm comprises an apparatus that cools the surface.

15. The robot of claim 13, further comprising:
at least one optical sensor associated with the arm and configured to sense the surface or a region thereof; and
an image-processing unit communicating with the at least one optical sensor.

16. The robot of claim 13, wherein at least one of the wavelength or an intensity of the emitted radiation is adjustable based on a pigmentation of at least one of the surface, the structure, or the at least one mark.

17. The robot of claim 13, further comprising:
a tactile sensor extending from the arm;
wherein the control device is further configured to receive signals from the tactile sensor, and to operate the drives to adjust or control movement of the arm based on the signals.

18. The robot of claim 13, wherein the radiation source comprises at least one laser from the group of a Ruby Laser (694.3 nm), an Nd:YAG Laser (1064 nm, frequency-doubled 532 nm), an Er:YAG Laser (2940 nm), an Alexandrite Laser (755 nm), or a CO₂ Laser (1064 nm, 9400 nm).

19. A method for machining or treating a surface that has at least one of a structure or at least one mark, the method comprising:
obtaining a robot comprising an arm having a plurality of links and a plurality of drives for moving the links, a processing head connected to the arm and comprising a radiation source that emits radiation, and a control device configured to actuate the drives;
operating the drives with the control device so as to adjust forces and/or torques applied by the drives such that the processing head or a first element that extends from the arm touches the surface and is moved along the surface while simultaneously stretching or tightening the surface;
emitting radiation from the processing head; and
adjusting at least one of a wavelength or intensity of the emitted radiation based on at least one of the structure or the at least one mark.

20. The method of claim 19, further comprising:
sensing at least one of the structure or the at least one mark with a sensor;
wherein adjusting at least one of a wavelength or intensity of the emitted radiation comprises adjusting based on at least one of the sensed structure or the at least one sensed mark.

21. The method of claim 19, further comprising:
sensing at least one of the structure or the at least one mark with a sensor; and
adjusting or controlling at least one of a movement, a speed, or an acceleration of the arm based on at least one of the sensed structure or the at least one sensed mark.

22. The method of claim 19, wherein the arm is provided with a plurality of processing heads, each configured to emit a different type of radiation, the method further comprising:
emitting radiation from at least one of the plurality of processing heads based on at least one of the structure or the at least one mark.

23. The method of claim 19, further comprising:
operating the drives with the control device to apply a pressure to the surface while the surface is machined or treated;
wherein the applied pressure is in the range of about 1 kPa and about 6 kPa.

24. The method of claim 19, wherein emitting radiation from the processing head comprises emitting laser radiation having an energy density in the range of 300 mJ/cm² and 10 J/cm² toward the surface.

25. The method of claim 24, wherein emitting radiation from the processing head comprises emitting laser radiation having an energy density in the range of 300 mJ/cm² and 2 J/cm² toward the surface.
